(19) United States
(12) Patent Application Publication
(10) Pub. No.: US 2010/0174309 A1
(43) Pub. Date: Jul. 8, 2010

(54) RECANALIZATION/REVASCULARIZATION AND EMBOLUS ADDRESSING SYSTEMS INCLUDING EXPANDABLE TIP NEURO-MICROCATHETER

(75) Inventors: JOHN FULKERSON, Rancho Santa Margarita, CA (US); David A. Ferrera, Redondo Beach, CA (US); Andrew Cragg, Edina, MN (US)

Correspondence Address: Luce, Forward, Hamilton & Scripps LLP, 2050 Main Street, Suite 600, Irvine, CA, 92614, US

(73) Assignee: MINDFRAME, INC., Orange County, CA (US)

(21) Appl. No.: 12/651,353

(22) Filed: Dec. 31, 2009

Related U.S. Application Data
(63) Continuation-in-part of application No. 12/123,390, filed on May 19, 2008.

(30) Foreign Application Priority Data
Nov. 12, 2009 (US) PCT/US2008/083185
Dec. 21, 2009 (GB) 0922251.4

(51) Int. Cl. A61B 17/22

(52) U.S. Cl. 606/200

(57) ABSTRACT

An acute stroke recanalization systems and processes include catheter-based improved reconstrainable or tethered neurological devices which are deliverable through highly constricted and tortuous vessels, crossing the zone associated with subject thrombi/emboli, where deployment impacts, addresses or bridges the embolus, compacting the same into lumenal walls which enables perfusion and lysis of the embolus, while the improved neurological medical device itself remains contiguous with the delivery system acting as a filter, basket or stand alone alternate medical device, depending on the status of the embolus and other therapeutic aspects of the treatment being offered for consideration.

### RELATED APPLICATION

This application claims the full Paris Convention benefit of and priority to U.S. Provisional Application Ser. No. 60/980,736, filed Oct. 17, 2007; is a continuation-in-part of U.S. patent application Ser. No. 12/123,390, filed May 19, 2008; UK patent application no. 0922251.4, which is a national stage entry of PCT/US2008/083185, the contents of each being incorporated by reference herein in their entirety, as if fully set forth herein.

### BACKGROUND

The present disclosure relates to minimally invasive and catheter delivered revascularization systems for use in the vasculature, especially those suited for usage above the juncture of the Subclavian Artery and Common Carotid Artery. In particular, this disclosure relates to revascularization devices for use in treatment of acute ischemic stroke, including improved neurological medical devices which are tethered or reconstrainable self-expanding neurological medical devices.

### SUMMARY

According to embodiments of the present invention, there are disclosed acute stroke revascularization/recanalization systems comprising, in combination; catheter systems having guidewires to access and emplace improved neurological medical devices into the cerebral vasculature, the systems including proximal stainless steel pushers with distal nitinol devices.

According to embodiments, there are disclosed one-piece nitinol devices in combination with the above disclosed and/or claimed catheter systems.

Briefly stated, according to embodiments a novel enhanced tethered revascularization device is deliverable through highly constricted and tortuous vessels, entering a zone associated with subject thrombi/emboli, where deployment impacts the embolus, compacting the same into luminal walls which enables perfusion and lysis of the embolus, while the revascularization device itself remains continuous with the delivery system acting as a filter, basket or stand alone revascularization mechanism, depending on the status of the embolus and other therapeutic aspects of the treatment being offered for consideration.

According to embodiments of the system and processes of the present invention, in certain iterations, once deployed the instant system compacts the embolus against the luminal wall, creating a channel for blood flow which may act like a natural lytic agent to lyse or dissolve the embolus.

According to embodiments, there is provided an improved neurological medical device which comprises, in combination, a catheter system effective for delivering a combination radial filter/revascularization device and basket assembly into a desired location in the cerebral vascular system, a self-expanding radial filter/revascularization device and basket assembly detachably tethered to the catheter system which functions in at least three respective modes, wherein the radial filter/revascularization device and basket assembly is attached to the catheter and wherein radial filter/revascularization device and basket assembly further comprises at least two states per mode, a retracted state and an expanded state; and wherein the radial filter/revascularization device and basket assembly may be retracted into the retracted state after deployment in an expanded state, in each mode.

According to embodiments, there is provided a process comprising in combination providing a revascularization device tethered to a catheter by emplacing the system into a patient for travel to a desired location in a vessel having an obstruction/lesion and deploying the revascularization device by allowing it to move from a first state to a second state across a lesion which compresses the subject embolus into a luminal wall to which it is adjacent whereby creating a channel for blood flow as a lytic agent, and removing the system which the obstruction/lesion is addressed.

It is noted that if blood flow does not lyse the blood embolus, lytic agents can be administered via the guidewire lumen, as a feature of the present invention.

According to embodiments, there is provided a process whereby the revascularization device tethered to a catheter functions as a radial filter to prevent downstream migration of emboli.

The U.S. Food and Drug Administration (FDA) has previously approved a clot retrieval device (The Merci® brand of retriever X4, X5, X6, L4, L5 & L6: Concentric Medical, Mountain View, Calif.). Unfortunately, when used alone, this clot retriever is successful in restoring blood flow in only approximately 50% of the cases, and multiple passes with this device are often required to achieve successful recanalization. IA thrombolytics administered concomitantly enhance the procedural success of this device but may increase the risk of hemorrhagic transformation of the revascularization infarction due to the mechanism of action of the Merci retrievers and length of time required to recanalize the veseel. There have been several reports of coronary and neuro-stent implantation used for mechanical thrombolysis of recalcitrant occlusions. In summary, stent placement with balloon-mounted or self-expanding coronary and neuro-types of stents has been shown to be an independent predictor for recanalization of both intracranial and extra cranial cerebro-vasculature occlusions. This provides some insight into approaches needed to overcome these longstanding issues.

By way of example, self-expanding stents designed specifically for the cerebro-vasculature can be delivered to target areas of intracranial stenosis with a success rate of >95% and an increased safety profile of deliverability because these stents are deployed at significantly lower pressures than balloon-mounted coronary stents. However, systems using this data have yet to become commercial, available or accepted by most practitioners.

The use of self-expanding stents is feasible in the setting of symptomatic large vessel intracranial occlusions. With stent placement as a first-line mechanical treatment or as a “last-resort” maneuver, TIMI/TICI 2 or 3 revascularization can be successfully obtained, according to clinical data now available.

The literature likewise suggests that focal occlusions limited to a single large vessel, particularly solitary occlusions of the MCA, ICA or Vertebral and Basilar Arteries, may be preferentially amenable to stent placement and thus can help clinicians to achieve improved rates of recanalization. In addition, there's a predominance of ischemic strokes in females but gender doesn't appear to play a role in the success of self-expanding stent implementation. However, systems need to be designed to confirm this.

Despite use of prourokinase rt-PA (recombinant tissue plasminogen activator) in the late 90's and increasing use of other antithrombotic agents (eg, Alteplase® and Reteplase®), recanalization rates remain approximately 60%. The major concerns with pharmacologic thrombolysis (alone) has been the rate of hemorrhage, inability to effectively lyse fibrin\platelet-rich clots, lengthy times to recanalization, and inability to prevent abrupt reocclusions at the initial site of obstruction. In PROACTII, ICH with neurologic deterioration within 24 hours occurred in 10.9% of the prourokinase group and 3.1% of the control group (P=0.06), without differences in mortality. Abrupt reocclusions or recanalized arteries has been found to occur relatively frequently, even with the addition of angioplasty or snare manipulation for mechanical disruption of thrombus, and seems to be associated with poor clinical outcomes.

The use of other mechanical means has been reported to be effective in recanalization of acute occlusions. It makes sense that a combination of mechanical and pharmacologic approaches would yield greater benefit.

A known investigation in an animal model has shown, both the Wingspan® brand of self-expanding stent and Liberté® brand of balloon-mounted stent (Boston Scientific, Boston, Mass.) were able to re-establish flow through acutely occluded vessels. The self-expanding stents performed better than the balloon-mounted stents in terms of navigability to the target site. The self-expanding stents incurred lower rates of vasospasm and side-branch occlusions, which suggests superiority of these stents, over balloon-mounted stents, to maintain branch vessel patency during treatment of acute vessel occlusion. In previous animal studies conducted, intimal proliferation and loss of lumen diameter were seen after the implantation of bare-metal, balloon-expandable stents. The literature further supports this set of issues.

These phenomena are believed to be attributable to intimal injury created during the high-pressure balloon angioplasty that is required for stent deployment.

Compared with coronary balloon-mounted stents, self-expanding stents designed for use in the intracranial circulation are superior because they are easier to track to the intracranial circulation and safer to deploy in vessels in which the true diameter and degree of intracranial atherosclerotic disease are unclear.

Moreover, based on previous experience, currently available self-expanding stents provide enough radial outward force at body temperature to revascularize occluded vessels, with low potential for the negative remodeling and in-stent restenosis that are associated with balloon-mounted stents in nonintracranial vascular beds.

Because self-expanding stents are not mounted on balloons, they are the most trackable of the stents currently available for the intracranial circulation. Unlike clot retrievers, which lose access to the target (occlusion site) every time they are retrieved (and often to necessitate multiple passes), self-expanding stents allow for wire access to the occlusion at all times, increasing the safety profile of the procedure by not requiring repeat maneuvers to gain access to the target site (as in the case for the Merci® brand of clot retriever).

Self-expanding stent placement of acute intracranial vessel occlusions may provide a novel means of recanalization after failure of clot retrieval, angioplasty, and/or thrombolytic therapy. The patency rates in this series are encouraging, yet issues remain to be addressed.

In the setting of acute stroke, restoring flow is of singular importance. In-stent stenosis or delayed stenosis may be treated in a delayed fashion on an elective basis, should the patient achieve a functional recovery from the stroke.

Recanalization with self-expanding stents may provide flow through the patent artery, and restore flow to the perforators, or, alternatively, they may remain occluded. Restoring flow to the main artery, however, will reduce the stroke burden. What is needed is a solution leveraging positive aspects of stent-based treatment without the negative outcomes which have been associated with traditional stenting.

### DRAWINGS

The above-mentioned features and objects of the present disclosure will become more apparent with reference to the following description taken in conjunction with the accompanying drawings wherein like reference numerals denote like elements and in which:

FIG. 1 is a perspective view of an embodiment of an acute stroke recanalization system according to embodiments of the present disclosure in a first configuration;

FIG. 2 is a perspective view of an embodiment of an acute stroke recanalization system according to embodiments of the present disclosure tailored for use with the neurovasculature in a second configuration, further illustrating modular aspects of the system as used with tethered or reconstrainable self-expanding neurological medical devices;

FIG. 3a shows a stroke device in cross section having an unexpanded state and an expanded state;

FIG. 3b shows a stroke device in cross section having a first state and a second state under pinching load;

FIG. 4 shows a cell of a stroke device with a portion in an expanded view;

FIG. 5 shows a stroke device with a cell thereof in an expanded view;

FIG. 6 shows a chart of chronic outward force of various devices, with force per unit length (N/mm) at given diameters (mm);

FIG. 7 shows a chart of radial resistive force of various devices, with force per unit length (N/mm) at given diameters (mm);

FIGS. 8A, 8B, 9A, 9B, 10A, 10B, 11A, and 11B show a variety of cell sizes and geometries that may be provided to achieve desired outcomes during therapy;

FIGS. 12, 13A, 13B, and 13C show a variety of individual cell sizes, with emphasis;

FIG. 14 shows a perspective view of a stroke device;

FIG. 15 shows a side view of a stroke device;

FIG. 16 shows a top view of a stroke device; and

FIG. 17 shows a front view of a stroke device.

### DETAILED DESCRIPTION

The present inventors have realized that by leveraging a conventional self-expanding revascularization device delivery platform, a poly-modic system can be iterated which impacts, addresses and/or crosses an embolus, radially filters, and either removes the offending embolus or is optionally emplaced to address the same. A paucity of extant systems effective for such combination therapies is noted among the art.

Using endovascular techniques self-expandable tethered or reconstrainable self-expanding neurological medical devices offer instant revascularization/recanalization of MCA's and related vessels, without any of the traditional concerns associated with stenting, according to embodiments of the present invention. It is likewise offered for consideration that conventional stenting devices, systems, and methods, on balance, have become known to have deleterious impacts on the cerebral vasculature often out-weighing specific therapeutic benefits of the same.

Expressly incorporated herein by reference are the following U.S. Letters patents and publications, each as if fully set forth herein: 2005/0119684; 2007/0198028; 2007/0208367; 2009/0125053; 2009/0105722; U.S. Pat. Nos. 5,449,372; 5,485,450; 5,792,157; 5,928,260; 5,972,019; 6,485,500; 7,147,655; 7,160,317; 7,172,575; 7,175,607; and 7,201,770.

The instant system allows for natural lysis, revascularization of the challenged vessels, and importantly radially filters any particulates generated, to obviate the need to be concerned with distal migration of the same, unlike prior systems or applications which include largely “off-label” usages of devices approved only for aneurysms in the brain, or mis-matched stenting endeavors which also create issues.

The present disclosure relates to revascularization devices used to treat, among other things, ischemic stroke. Naturally, therefore, the revascularization devices of the present disclosure are designed to be used in neuro-type applications, wherein the specifications of the present catheters and revascularization devices may be deployed in the blood vessels of the cerebral vascular system. Similarly contemplated for the revascularization systems and catheters of the present disclosure is deployment in other parts of the body wherein the specifications of the present disclosure may be used in other vessels of the body in a non-invasive manner.

According to embodiments, disclosed herein is a catheter-based revascularization system. The revascularization devices of the present disclosure are for revascularization of blood vessels. When the catheter-based revascularization system of the present disclosure is deployed into a blood vessel having an embolus, the revascularization device is expanded thereby opening the vessel so that the vessel can resume proper blood flow.

According to the instant teachings, deployment of the system of the present disclosure establishes immediate approximately 50% of the diameter of the lumen patency of the vessel being addressed. Among the prior art, no system having adequately small profile with flexibility to promote improved access for in-site treatment is known which may be used as a temporary (not implanted) solution. Those skilled in the art readily understand that detachment methods comprising mechanical, electrical, hydraulic, chemical, or thermal, and others are within the scope of the instant teachings.

Moreover, as the embolus lyses, either via blood flow or by infusing lytic agents than the guidewire lumen, the deployed revascularization device radially filters larger embolus particles from traveling downstream, thereby reducing the chances of further complications. Once the blood vessel is revascularized, the revascularization device is modified to be in a removable state together with filtered detritus, and the catheter-revascularization system is removed from the blood vessels of the patient.

Likewise, in the event that no resolution of the embolus is noted in the instant revascularization system the inventors contemplate detachment and employment as a stent of the cage-like membrane. Angiographic recanalization has been associated with improvement in clinical outcome in the setting of acute stroke resulting from acute intracranial thrombotic occlusion. Anatomic limitations (tortuous anatomy, length of the occlusion, or location of occlusion) or supply limitations are among the reasons precluding use of prior art systems until the adverse of the instant teachings.

Stenting has been used successfully to restore flow after abrupt reocclusion occurring after recanalization with other modalities in previous cases. Stenting has also been reported in cases in which other modalities have failed to recanalize vessels. Even if an underlying stenosis is rarely the cause of stroke, stenting may play a role by morselizing the embolic clot or trapping it against the arterial wall.

In spite of attendant risks, the literature suggests that the use of intracranial stents as a method for arterial recanalization during cerebral ischemia caused by focal occlusion of an intracranial vessel has been demonstrated to have benefits in some cases. Despite the use of available pharmacological and mechanical therapies, angiographic recanalization of occluded vessels has not been adequately achieved before stent placement, in most cases. This underscores the need for the present invention.

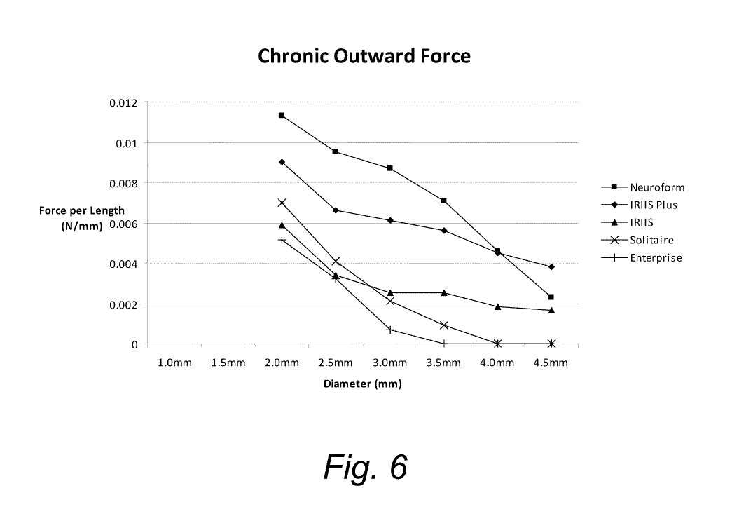

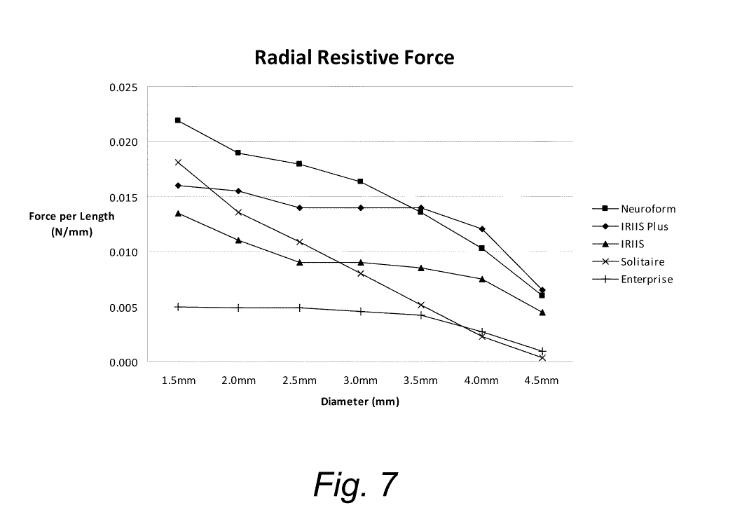

When SAH and intracranial hematoma occurred in patients in whom balloon-mounted stents were used, they most likely resulted from distal wire perforation. The distal wire purchase needed to navigate a coronary stent into the intracranial circulation may explain the occurrence of these adverse events. Alternatively, multiple manipulations of the Merci® brand of retriever device or expansion of balloon-mounted stents may have induced microdissections in the vessel. Stents designed for intracranial navigation have better navigability and pliability. The Wingspan® brand of stent (Boston Scientific) was designed to have more radial force than the Neuroform® brand of stent and may further improve this technique. However, the art clearly needs to advance further in this area, as supported herein in FIGS. 6 and 7, inter alia.

IA therapy for stroke has evolved during the past decade. Approval of the Merci® brand of retriever device represents a significant step toward achieving better outcomes in acute stroke for patients not suitable for or refractory to IV tPA. However, recanalization is not always achieved using this device. Therefore, additional treatment options are required, as offered for consideration herein.

Spontaneous dissection of the internal carotid artery (ICA) is one of the main causes of ischemic stroke in young and middle-aged patients, representing 10% to 25% of such cases. Because infarct due to dissection is mainly thromboembolic, anticoagulation has been recommended to prevent new stroke in patients with acute dissection, provided they have no contraindications. In the acute phase, intravenous recombinant tissue-type plasminogen activator (IV rtPA) given within 3 hours after onset of stroke due to dissection is reportedly safe and effective. However, this often needs supplemental therapy to be effective.

Endovascular treatment with stent deployment for ICA dissection with high-grade stenosis or occlusion may be most appropriate when anticoagulation fails to prevent a new ischemic event. In such cases, the MCA may be patent. However, to compare outcomes of patients with acute stroke consecutive to MCA occlusion due to ICA dissection treated either by stent-assisted endovascular thrombolysis/thrombectomy or by IV rtPA thrombolysis. Stent assisted endovascular thrombolysis/thrombectomy compared favorably with IV rtPA thrombolysis, underscoring the need for the instant device.

The main limitation of this procedure is the immediate need for an experienced endovascular therapist. The number of cases of MCA occlusion due to carotid artery dissection was quite small and represented <10% of patients admitted for carotid dissection. However, despite these promising preliminary results, potential drawbacks related to the procedure must be considered. Acute complications such as transient ischemic attack, ischemic stroke, femoral or carotid dissection, and death have been reported. Other potential hazards of endovascular treatment of carotid dissection could have been observed. On balance, the risk-benefit favors solutions like the present invention.

Most patients with acute cerebrovascular syndrome with MCA occlusion consecutive to ICA dissection have poor outcomes when treated with conventional IV rtPA thrombolysis, whereas most patients treated with stent-assisted endovascular thrombolysis/thrombectomy show dramatic improvements. Further large randomized studies are required to confirm these data, which trends likewise are technical bases for the instant systems.

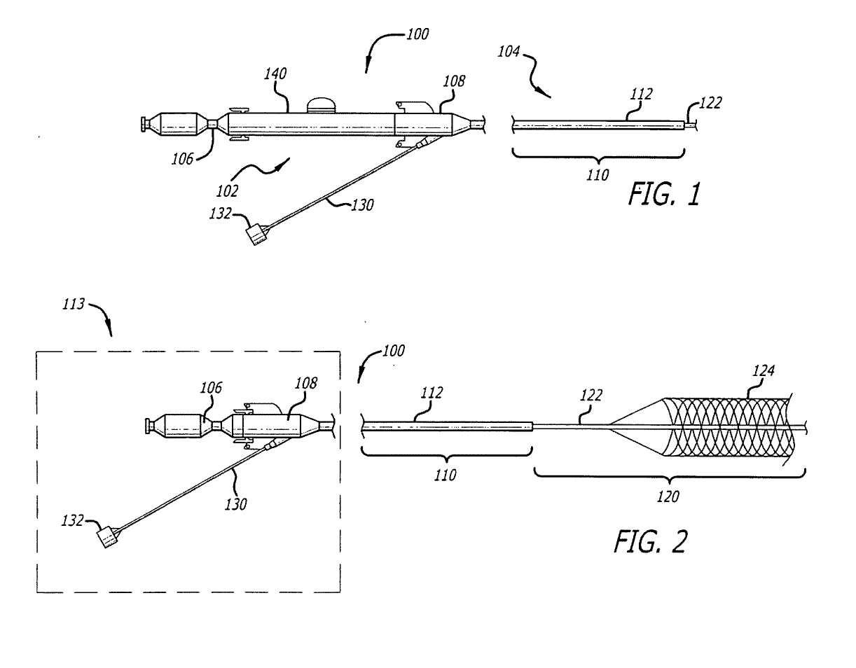

According to embodiments and as illustrated in FIG. 1, catheter-based revascularization system 100 provides a platform for lysing emboli in occluded blood vessels. Accordingly, catheter-based revascularization system 100 generally comprises control end 102 and deployment end 104. According to embodiments, control end 102 is a portion of the device that allows a user, such as a surgeon, to control deployment of the device through the blood vessels of a patient. Included as part of control end 102 is delivery handle 106 and winged apparatus 108, in some embodiments. Those skilled in the art readily understand module 113 (see FIG. 2) is detachable.

According to some examples of the instant system during shipping of catheter-revascularization system 100, shipping lock (not shown) is installed between delivery handle 106 and winged apparatus 108 to prevent deployment and premature extension of revascularization device 124 (see FIG. 2) while not in use. Furthermore, by preventing delivery handle 106 from being advanced towards winged apparatus 108, coatings applied to revascularization device 124 are stored in a configuration whereby they will not rub off or be otherwise damaged while catheter-based revascularization system 100 is not in use.

According to embodiments, agent delivery device 130 provides a conduit in fluid communication with the lumen of the catheter-based revascularization system 100 enabling users of the system to deliver agents through catheter-revascularization system 100 directly to the location of the embolus. The instant revascularization system delivery device may be made from materials known to artisans, including stainless steel hypotube, stainless steel coil, polymer jackets, and/or radiopaque jackets.

Accordingly, luer connector 132 or a functional equivalent provides sterile access to the lumen of catheter-based revascularization system 100 to effect delivery of a chosen agent. Artisans will understand that revascularization devices of the present invention include embodiments made essentially of nitinol or spring tempered stainless steel. Revascularization devices likewise may be coated or covered with therapeutic substances in pharmacologically effective amounts or lubricious materials. According to embodiments, coatings include nimodipene, vasodialators, sirolamus, and paclitaxel. Additionally, at least heparin and other coating materials of pharmaceutical nature may be used.

Deployment end 104 of catheter-based revascularization system 100 comprises proximal segment 110 and distal segment 120. Proximal segment 110, according to embodiments, houses distal segment 120 and comprises outer catheter 112 that is of a suitable length and diameter for deployment into the blood vessel of the neck, head, and cerebral vasculature. For example in some embodiments, proximal segment 110 is from at least about 100 cm to approximately 115 cm long with an outer diameter of at least about 2.5 French to about 4 French.

Referring also to FIG. 2, distal segment 120 comprises inner catheter 122 and revascularization device 124 (as shown here in one embodiment having uniform cells, variable cells likewise being within other embodiments of the present invention), which is connected to inner catheter 122. Inner catheter 122, according to embodiments, is made from stainless steel coil, stainless steel wire, or ribbon or laser cut hypotube and is of a suitable length and diameter to move through outer catheter 112 during deployment. For example, inner catheter 122 extends from outer catheter 112 38 cm, thereby giving it a total length of between at least about 143 and 175 cm. The diameter of inner catheter 122 according to the exemplary embodiment is 2.7 French, with an inner diameter of at least about 0.012 to 0.029 inches. The inner diameter of inner catheter 122 may be any suitable diameter provided inner catheter 122 maintains the strength and flexibility to both deploy and retract revascularization device 124.

Referring to both FIGS. 1 and 2, revascularization device 124 is a self-expanding, reconstrictable retractable device tethered to inner catheter 122. Revascularization device 124 may be made from nitinol, spring tempered stainless steel, or equivalents as known and understood by artisans, according to embodiments. Revascularization device 124, according to embodiments and depending on the particular problem being addressed, may be from at least about 3.5 mm to about 50 mm in its expanded state. In an expanded state, revascularization device 124 is designed to expand in diameter to the luminal wall of blood vessel where it is deployed.

As known to artisans, revascularization device 124 may be coated or covered with substances imparting lubricous characteristics or therapeutic substances, as desired. Naturally, the expandable mesh design of revascularization device 124 must by a pattern whereby when revascularization device 124 is retracted, it is able to fully retract into inner catheter 122. The nature of the cell type likewise changes with respect to the embodiment used, and is often determined based upon nature of the clot.

Catheter-revascularization system 100 is deployed through a patient's blood vessels. Once the user of catheter-revascularization system 100 determines that the embolus to be addressed is crossed, as known and understood well by artisans, revascularization device 124 is deployed by first positioning outer catheter 112 in a location immediately distal to the embolus.

Then, to revascularize/reperfuse the occluded blood vessel, distal catheter 120 is deployed in a location whereby revascularization device 124 expands at the location of the embolus, as illustrated by FIG. 2. The embolus is thereby compressed against the luminal wall of the blood vessel and blood flow is restored. Modular detachable segment 113 is known also, and may be swapped out, as needed, if an Rx system is used.

As discussed above and claimed below, creating a channel for flow ideally includes making a vessel at least about halfway-patent, or 50% of diameter of a vessel being open. According to other embodiments, the channel created may be a cerebral equivalent of thrombolysis in myocardial infarction TIMI 1, TIMI 2, or TIMI 3.

Restoration of blood flow may act as a natural lytic agent and many emboli may begin to dissolve. Revascularization device 124 is designed, according to embodiments, to radially filter larger pieces of the dissolving embolus and prevent them from traveling distal to the device and potentially causing occlusion in another location. Because the revascularization device provides continuous radial pressure at the location of the obstruction, as the embolus lyses, the blood flow continues to increase.

After the embolus is lysed, revascularization device 124 is resheathed into outer catheter 112 and removed from the body. According to embodiments, larger pieces of the thrombus may be retracted with revascularization device 124 after being captured in the radial filtering process. According to embodiments, revascularization device 124 may be detachable whereby the revascularization device 124 may detach from catheter-based revascularization system 100 if it is determined that revascularization device 124 should remain in the patient. As discussed above, illustrated in the Figures, and claimed below according to embodiments, catheter-based revascularization system 100 reconstrainable attachment or attachment by tether may be optionally detachable. Revascularization device detachment methods comprise mechanical, electrical hydraulic, chemical, thermal, and those other uses known to artisans.

According to embodiments of the present disclosure, clot therapy may have one or more of at least three objectives or effects: maceration of a clot, removal of a clot, and lysis of a clot.

Maceration of a clot refers to the process or result of softening of the clot or breaking the same into pieces mechanically or by using vascular fluids. For example, pressing or compressing the clot with a mechanical member can cause the clot to soften, break up or fragment, whereby, exposure of the clot (or portions thereof) to vascular flow may cause the clot (or portions thereof) to macerate, soften, or diffuse.

Removal of a clot refers to the process or result of relocating the clot or portions thereof. A variety of methods may be employed to remove a clot, according to the present disclosure.

Lysis of a clot refers to any chemical, biological, or other cellular or sub-cellular process or result of altering the structure of a clot. Lysis may refer to fibrinolysis—degradation of fibrin—within a fibrin clot by application of enzymes. For example, lysis may occur in the presence of plasmin, heparin, etc.; precursors or activation peptides thereof; or inhibitors of fibrin development.

According to embodiments, characteristics of stroke device 200 may be controlled to modify the effect of stroke device 200 to achieve one or more of maceration, removal, and lysis of a clot. For example, hoop strength, stiffness, cell size, strut length, strut width, and strut thickness of stroke device 200 may be varied to provide customizable therapies to a clot.

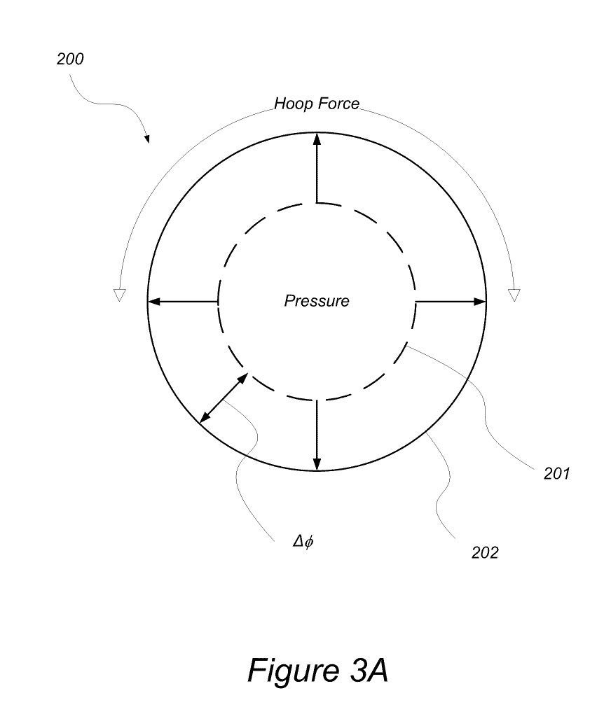

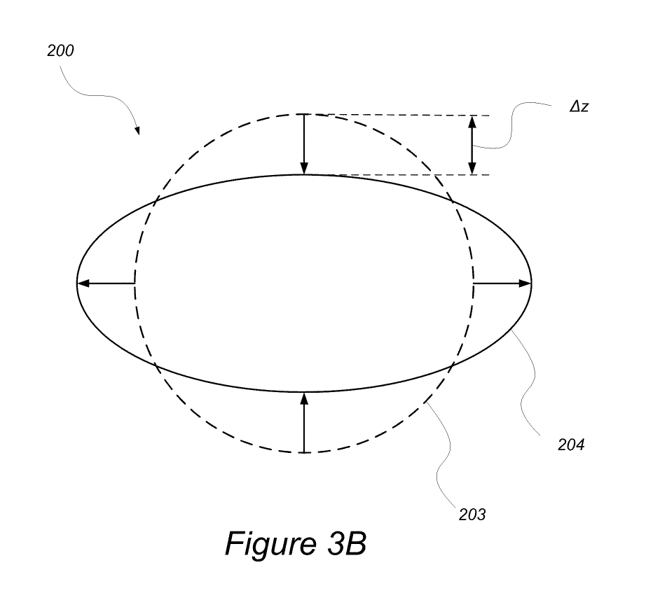

Blood vessels may experience loads from a variety of sources, such as the expansion of stroke device 200. Pressures applied to any cylindrical structure, such as a blood vessel, result in hoop, or circumferential loading of the vessel (FIG. 3A). Both the applied pressure and the resulting hoop stress have units of force per unit area, but these may differ in direction. As used herein, “pressure” refers to the force normal to the vessel wall, divided by the surface area of the lumen. As used herein, “hoop stress” is the circumferential load in the vessel wall divided by the cross-sectional area of the vessel wall (length times wall thickness).

The relationship between the pressure (p) and the hoop stress (σ) in a thin-walled cylindrical object, such as stroke device 200, may be expressed as:

$$σ = \frac{p φ}{2 t} , \qquad ( Eq . 1 )$$

where “φ” is the diameter of stroke device 200 and “t” is the wall thickness of stroke device 200. The hoop force (F_{θ}) in a vessel wall may be expressed as:

$${F}_{θ} = σ tL = \frac{p φ L}{2} , \qquad ( Eq . 2 )$$

where “L” is the length of stroke device 200 (or length “Lₛ” of strut 220, depending on the scope of analysis). The hoop force per unit length (f_{θ}) may be expressed as:

$${f}_{θ} = \frac{{F}_{θ}}{L} = σ t = \frac{p φ}{2} . \qquad ( Eq . 3 )$$

“Stiffness,” or the elastic response of a device to an applied load, reflects the effectiveness of stroke device 200 in resisting deflection due to vessel recoil and other mechanical events. Those skilled in the art will note that “stiffness” is the inverse of “compliance,” or diameter change (Δφ) at a specific applied pressure (p). As shown in FIG. 3A, stroke device 200 shown in cross section may experience a change in diameter (Δφ) as it expands from a compressed state 201 to an uncompressed state 202. The hoop stiffness (k_{θ}) of stroke device 200 may be expressed as the hoop force per unit length (f_{θ}) required to elastically change its diameter (Δφ), or:

$${k}_{θ} = \frac{{f}_{θ}}{Δ φ} . \qquad ( Eq . 4 )$$

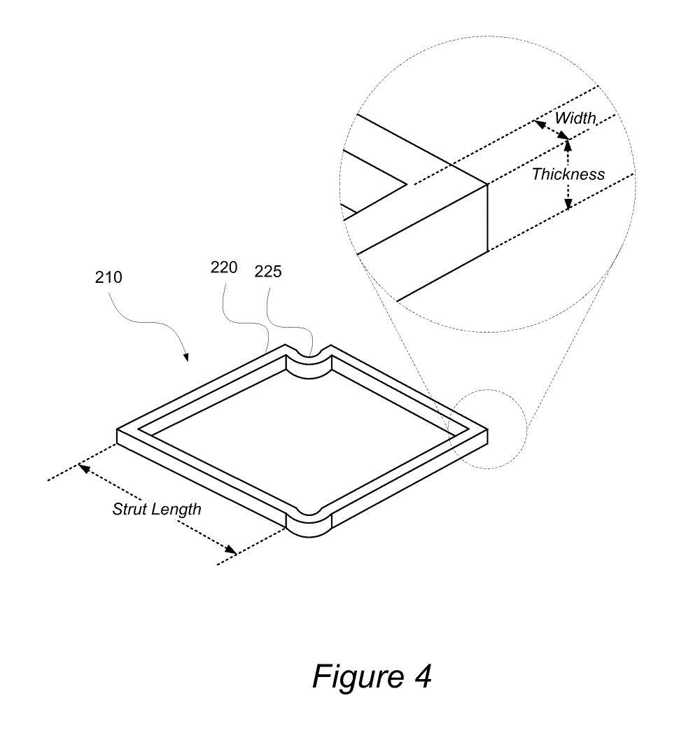

A change in diameter (Δφ) of stroke device 200 due to an applied load is related to the geometry of stroke device 200 as expressed by:

$$Δ φ ∝ \frac{f φ {nL}_{s}^{3}}{{Ew}^{3} t} . \qquad ( Eq . 5 )$$

where “Lₛ” is the length of a strut (as shown in FIG. 4), “w” is the strut width (as shown in FIG. 4), “t” is the thickness of stroke device 200 (as shown in FIG. 4), “n” is the number of struts around the circumference of stroke device 200, and “E” is the elastic modulus of the material. Combining Eq. 3 with Eq. 5, the change in diameter (Δφ) of stroke device 200 may be related to an applied pressure load (p) by:

$$Δφ ∝ \frac{p φ {nL}_{s}^{3}}{{Ew}^{3} t} , \qquad ( Eq . 6 )$$

Combining Eq. 4 and Eq. 6, the hoop stiffness (k₀) may be expressed as:

$${k}_{θ} ∝ \frac{{Ew}^{3} t}{{nL}_{s}^{3}} . \qquad ( Eq . 7 )$$

Thus, hoop stiffness (k_{θ}) has a cubic relationship with strut width (w), a linear relationship with strut thickness (t), an inversely linear relationship with number of struts about the circumference (n), and an inversely cubic relationship with the strut length (Lₛ).

In contrast to symmetrical radial expansion and compression, an uneven load (i.e., pinching load) may be applied to an external surface of a portion of stroke device 200, resulting in radially asymmetric deflection (Δz). For example, as shown in FIG. 3B, stroke device 200 may be squeezed between two opposite loads, whereby stroke device 200 is subjected to a pinching load. Under a pinching load, stroke device 200 may deflect from an initial state 203 to a deflected state 204. A pinching load may cause struts 220 to be bent in a manner other than about the circumference. Pinching stiffness (kₚ), or the force required to cause radially asymmetric deflection (Δz) may be generalized by the expression:

$${k}_{p} ∝ \frac{{Et}^{3} w}{{nL}_{s}^{3}} . \qquad ( Eq . 8 )$$

Under a pinching load, the pinching stiffness (kₚ) of stroke device 200 has a cubic relationship with strut thickness (t) and a linear relationship with strut width (w). This is relationship is the inverse of the strut's influence on hoop stiffness (k_{θ}). Thus, strut thickness (t) has a dominant role in pinching stiffness (kₚ) and strut width (w) has a dominant role in hoop stiffness (k_{θ}).

According to embodiments, a clot in an otherwise substantially radially symmetric vessel may tend to cause radially asymmetric deflection of a stroke device 200 as it is expanded against the clot. Both hoop stiffness (k_{θ}) and pinching stiffness (kₚ) of stroke device 200 play a role in how stroke device 200 interacts with the clot.

According to embodiments, for a given pressure provided by stroke device 200, a smaller strut width (w) increases the amount of pressure per unit area applied by stroke device 200. Thus, the struts 220 of stroke device 200 may more easily cut through a clot with a smaller strut width. According to embodiments, a larger strut width (w) improves channel development through a clot. Where a strut provides a wider width, it displaces a greater amount of clot against the walls of the blood vessel. For example, strut width of a stroke device may be from about 0.010 to 0.100 about microns.

According to embodiments, stroke device 200 may provide both a chronic outward force (“COF”) and a radial resistive force (“RRF”). As used herein, chronic outward force (“COF”) is the continuing radial opening force of a self-expanding stroke device 200 acting on a vessel wall after having reached equilibrium with the vessel wall. As used herein, radial resistive force (“RRF”) is the force generated by a self-expanding stroke device 200 to resist compression. Generally, RRF is expressed in relation to the amount of relative compression to be achieved.

According to embodiments, the COF of various vascular therapy devices are provided in FIG. 6. As shown in FIG. 6, the COF per unit length of each device (N/mm) is shown at each of a variety of diameters (mm). The devices shown are (a) IRIIS™ device (by MindFrame® of Lake Forest, Calif.), (b) Solitaire™ AB device (by ev3® of Plymouth, Minn.), (c) Enterprise™ device (by Cordis® of Bridgewater, N.J.), (d) NeuroForm³™ (by Boston Scientific® of Boston, Mass.) device, and (e) IRIIS Plus™ device (by MindFrame® of Lake Forest, Calif.).

According to embodiments, the RRF of various vascular therapy devices are provided in FIG. 7. As shown in FIG. 7, the RRF per unit length of each device (N/mm) is shown at each of a variety of diameters (mm). The devices shown are (a) IRIIS™ device, (b) Solitaire™ AB device, (c) Enterprise™ device, (d) NeuroForm³™ device, and (e) IRIIS Plus™ device.

According to embodiments, COF testing results taken include the following data of force per unit length (N/mm):

| Diameter | NeuroForm³ ™ | IRIIS ™ Plus | IRIIS ™ | Solitaire ™ AB | Enterprise ™ |
|---|---|---|---|---|---|
| 2.0 mm | 0.01130 | 0.0090 | 0.00590 | 0.00700 | 0.00517 |
| 2.5 mm | 0.00950 | 0.0066 | 0.00340 | 0.00410 | 0.00320 |
| 3.0 mm | 0.00870 | 0.0061 | 0.00255 | 0.00210 | 0.00068 |
| 3.5 mm | 0.00710 | 0.0056 | 0.00255 | 0.00090 | 0.00000 |
| 4.0 mm | 0.00460 | 0.0045 | 0.00185 | 0.00000 | 0.00000 |
| 4.5 mm | 0.00230 | 0.0038 | 0.00165 | 0.00000 | 0.00000 |

According to embodiments, RFF testing results taken include the following data of force per unit length (N/mm):

| Diameter | NeuroForm³ ™ | IRIIS ™ Plus | IRIIS ™ | Solitaire ™ AB | Enterprise ™ |
|---|---|---|---|---|---|
| 1.5 mm | 0.022 | 0.016 | 0.014 | 0.018 | 0.005 |
| 2.0 mm | 0.019 | 0.016 | 0.011 | 0.014 | 0.005 |
| 2.5 mm | 0.018 | 0.014 | 0.009 | 0.011 | 0.005 |
| 3.0 mm | 0.016 | 0.014 | 0.009 | 0.008 | 0.005 |
| 3.5 mm | 0.014 | 0.014 | 0.009 | 0.005 | 0.004 |
| 4.0 mm | 0.010 | 0.012 | 0.008 | 0.002 | 0.003 |
| 4.5 mm | 0.006 | 0.007 | 0.005 | 0.000 | 0.001 |

A table is provided below showing the average COF and RRF of each of the devices tested.

| | | IRIIS ™ | | Solitaire ™ | |
|---|---|---|---|---|---|
| | NeuroForm³ ™ | Plus | IRIIS ™ | AB | Enterprise ™ |
| Average COF per unit | 0.0073 | 0.0059 | 0.0030 | 0.0023 | 0.0015 |
| length (N/mm) (across 2.0 mm | | | | | |
| to 4.5 mm diameter) | | | | | |
| Average RRF per unit | 0.0138 | 0.0127 | 0.0083 | 0.0067 | 0.037 |
| length (N/mm) (across 2.0 mm | | | | | |
| to 4.5 mm diameter) | | | | | |

According to embodiments, stroke device 200 having relatively low COF and RRF is effective for facilitating maceration of a clot. For example, the range of COF and RRF provided by the Mindframe IRIIS device offers effective therapy requiring maceration of a clot.

According to embodiments, stroke device 200 having relatively high COF and RRF is effective for facilitating removal of a clot. For example, the range of COF and RRF provided by the Mindframe IRIIS Plus device offers effective therapy requiring removal of a clot.

According to embodiments, stroke device 200 may have a range of COF per unit length across given diameters. For example, COF may be from about 0.00590 N/mm to about 0.0090 N/mm at a diameter of about 2.0 mm and a COF from about 0.00165 N/mm to about 0.0038 N/mm at a diameter of about 4.5 mm. It is noteworthy that at such expanded states, stroke device 200 may provide a non-zero force.

According to embodiments, stroke device 200 may have a range of RRF per unit length across given diameters. For example, RRF may be from about 0.011 N/mm to about 0.016 N/mm at a diameter of about 2.0 mm and a RRF from about 0.005 N/mm to about 0.007 N/mm at a diameter of about 4.5 mm.

According to embodiments, stroke device 200 may have an average COF per unit across a diameter of 2.0 mm to 4.5 mm length across a diameter of 2.0 mm to 4.5 mm of between about 0.0023 N/mm and about 0.0073 N/mm, more specifically between about 0.0030 N/mm and about 0.0059 N/mm. According to embodiments, stroke device 200 may have an average RRF per unit length across a diameter of 2.0 mm to 4.5 mm of between about 0.0067 N/mm and about 0.0138 N/mm, more specifically between about 0.0083 N/mm and about 0.0127 N/mm. Therapy provided within these ranges may provide effective maceration toward the lower end of the range and effective removal toward the upper end of the range.

According to embodiments, strut thickness of stroke device 200 may be from about 40 microns to about 60 microns. Strut width may be from about 50 microns to about 60 microns (for example, about 54 microns).

Traditionally, in many stents and stent-like structures, one goal is to achieve a ratio of strut thickness to strut width of at least 1.4. Such high ratios have been traditionally preferred for sustaining long-term emplacement of the device. As those with skill in the art will recognize, a ratio of 1.4 or greater aides in the performance of the structure by guiding the manner in which the struts bend. By providing the struts with more thickness than width, the structure innately “knows” how to bend and load the struts. With such characteristics, the device is easier to manufacture because it improves shape setting, the device crimps better, and the device is better able to resist loading that is normal to diameter.

Because pinching stiffness (kₚ) is predominantly determined by strut thickness and hoop stiffness (k_{θ}) is predominantly determined by strut width, a structure with a relatively high ratio of strut thickness to strut width will provide relatively high pinching stiffness (kₚ). In other words, given a thickness to width ratio of at least 1.4, the pinching stiffness of the device increases rapidly when greater hoop stiffness are desired. For example, to increase the hoop stiffness at a certain rate to achieve desired hoop stiffness characteristics would cause pinching stiffness to increase by at least about double the rate at which the hoop stiffness is increased for ratios exceeding 1.4. These increases in pinching stiffness may result in undesirable characteristics of the resulting structure. In contrast, a structure with a relatively low ratio of strut thickness to strut width will provide relatively high hoop stiffness (k_{θ}) without yielding detrimentally rapid increases in pinching stiffness.

According to embodiments, stroke device 200 of the present disclosure may have a strut thickness to strut width ratio of less than at least about 1.1, 1.2, 1.3, 1.4, or 1.5, etc. For example, The ratio of strut thickness to strut width may be between about 0.7 to about 1.2. Stroke device 200 of the present disclosure may achieve this strut thickness to strut width ratio of less than 1.4 due to dimensional constraints. For example, stroke device 200 may achieve lower ratios where it is applied for temporary or short-term therapy rather than permanent or long-term emplacement.

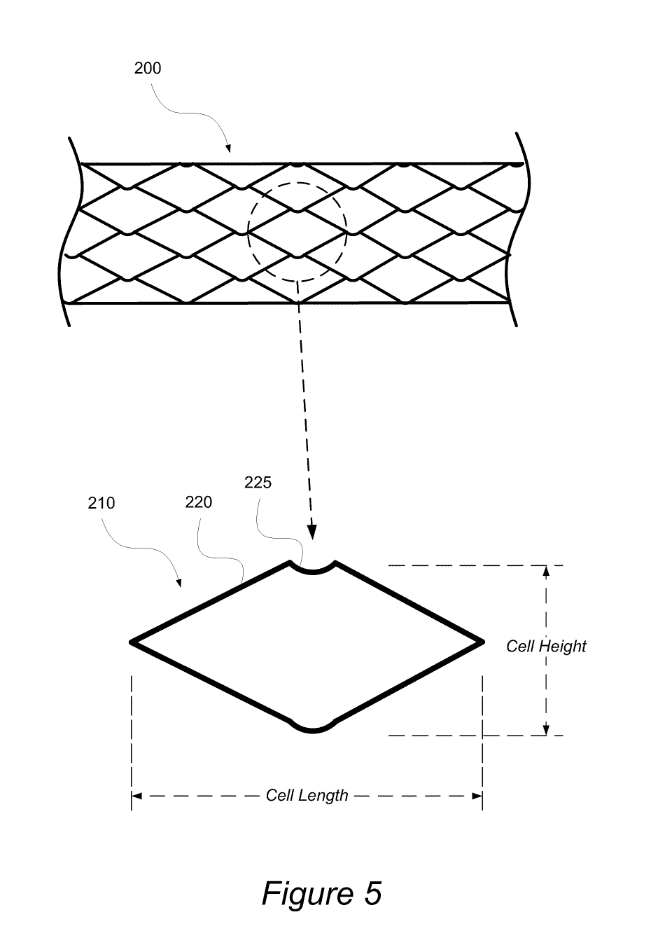

According to embodiments, cell size contributes to the effect that stroke device 200 has on a clot. As shown in FIG. 5, each open cell 210 of stroke device 200 may have a cell height and cell length, providing exposure from an interior portion of stroke device 200 to an exterior portion of stroke device 200. The cells 210 of stroke device 200 may include struts 220 and bridges 225 connecting struts 220. Bridges 225 may be of a variety of shapes and sizes, including “C” shapes, “S” shapes, straight shapes, etc. Cells 210 may form a variety of shapes, including diamonds, rectangles, and other polygonal shapes.

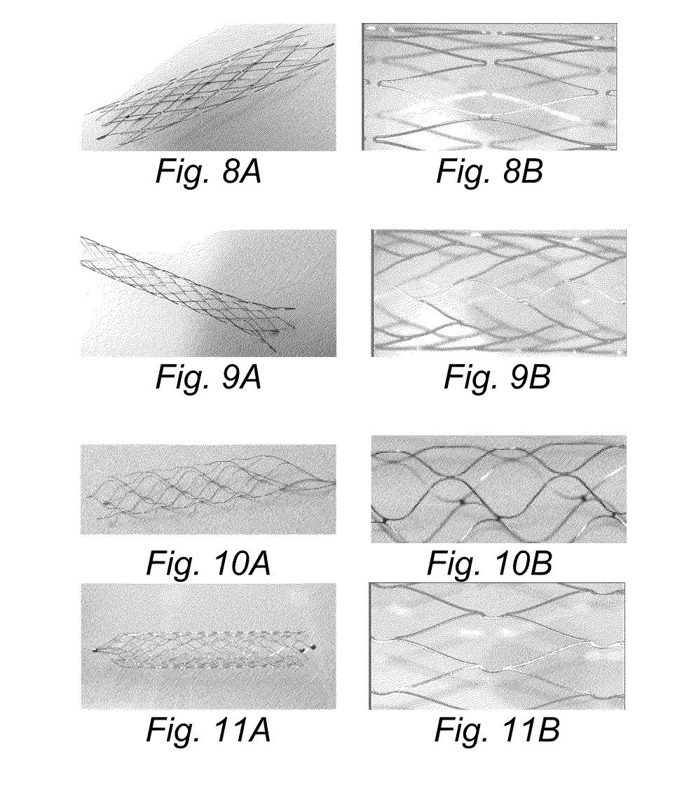

According to embodiments, as shown in FIGS. 8A, 8B, 9A, 9B, 10A, 10B, 11A, and 11B, a variety of cell sizes and geometries may be provided to achieve desired outcomes during therapy. FIGS. 8A and 8B show a NeuroForm³™ (by Boston Scientific® of Boston, Mass.) device. FIGS. 9A and 9B show a Enterprise™ device (by Cordis® of Bridgewater, N.J.). FIGS. 10A and 10B show a Solitaire™ AB device (by ev3® of Plymouth, Minn.). FIGS. 11A and 11B show a IRIIS™ device (by MindFrame® of Lake Forest, Calif.).

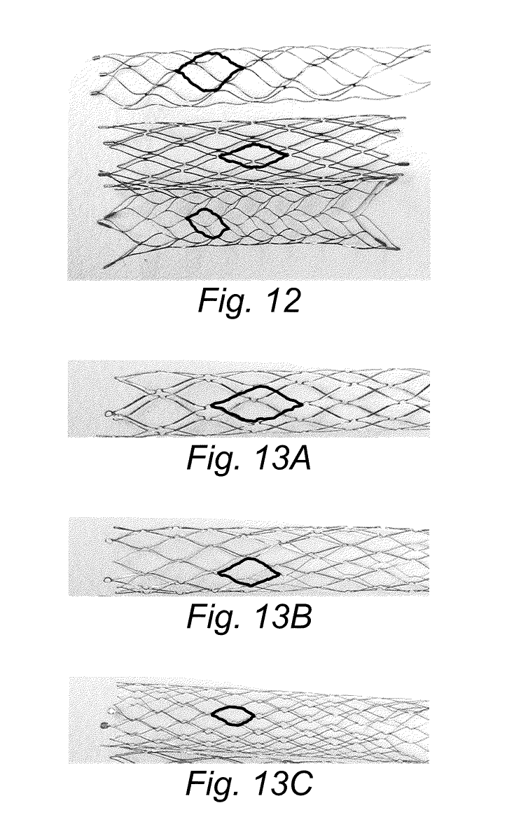

As shown in FIGS. 12, 13A, 13B, and 13C, individual cells 210 are shown with emphasis. FIG. 12 show views of each of a Solitaire™ AB device, a NeuroForm³™ device, and a Enterprise™ device. FIGS. 13A, 13B, and 13C each show an IRIIS™ device. The respective cell sizes of each are shown with emphasis. In particular, FIGS. 13A, 13B, and 13C show similar cell geometries with distinct cell sizes and the impact on the overall structure of the respective device. A relatively larger cell size is shown in FIG. 13A, with a relatively smaller cell size shown in FIG. 13C and an intermediate cell size shown in FIG. 13B.

Provided below is a table comparing characteristics of various vascular devices:

| | | | | | | IRIIS ™ |
|---|---|---|---|---|---|---|
| | | IRIIS ™ | | Solitaire ™ | | Large |
| | NeuroForm³ ™ | Plus | IRIIS ™ | AB | Enterprise ™ | Cell |
| Strut | 0.0065″ | 0.0024″ | 0.0027″ | 0.0035″ | 0.0027″ | 0.0024″ |
| Thickness | | | | | | |
| (inches) | | | | | | |
| Cell Size | 0.200″ × 0.070″ | 0.120″ × 0.050″ | 0.120″ × 0.050″ | 0.230″ × 0.200″ | 0.100″ × 0.050″ | 0.250″ × 0.100″ |
| (inches) | | | | | | |
| Cell Area | 0.007 | 0.003 | 0.003 | 0.023 | 0.0025 | 0.0250 |
| (sq. inches) | | | | | | |

According to embodiments, stroke device 200 having a larger cell size facilitates removal of a clot by allowing larger portions of the clot to be isolated as the closed portions (e.g., struts) of the cells apply pressure and force to the clot. The larger cell sizes cause larger portions of the clot to remain, whereby the relatively larger portions may be more readily captured and removed with stroke device 200 or other devices. Variation of radial strength can affect removal characteristic such as the ability to navigate through the intracranial vessel tortuosity.

According to embodiments, stroke device 200 having a small cell size facilitates lysis of a clot by breaking the clot into smaller portions. The smaller cell sizes cause smaller portions of the clot to remain, whereby more surface area of the clot is exposed to ambient materials for facilitating lysis. Variation of the cell size may affect clot lysis by varying the amount of surface area applying pressure from the structure to the clot. For example, smaller cell sizes will generally provide a greater amount of structure to transfer pressure and forces to a clot. Furthermore, a structure having smaller cells may provide a more consistently shaped channel (with fewer or less dramatic inflection points) for recanalization by more evenly distributing the outward forces and pressures. The improved recanalization in turn facilitates improved lysis by virtue of better exposure of the clot to vascular flow.

According to embodiments, stroke device 200 may have cells 210 of cell length from at least about 0.120″ to at least about 0.250″. According to embodiments, stroke device 200 may have cells 210 of cell height from about 0.050″ to about 0.100″. For example, stroke device 200 having cells 210 of cell length of about 0.120″ and cell height of about 0.050″ may be effective for macerating a clot to which stroke device 200 is applied. By further example, stroke device 200 having cells 210 of cell length of about 0.250″ and cell height of about 0.100″ may be effective for removing a clot to which stroke device 200 is applied.

According to embodiments, the cell height and cell length of each cell may yield an area defined by the boundaries of the cell. For example, stroke device 200 may have cells each having an area of between about 0.006 sq. inches to about 0.025 sq. inches. More specifically, of each cell may yield an area defined by the boundaries of the cell. For example, stroke device 200 may have cells each having an area of between about 0.010 sq. inches to about 0.020 sq. inches. According to embodiments, stroke device 200 having small cells 210 and high radial strength provides better channel development and maceration with relatively softer clots. According to embodiments, stroke device 200 having larger cells and high radial strength will provide better maceration and retrieval for firm, white clots.

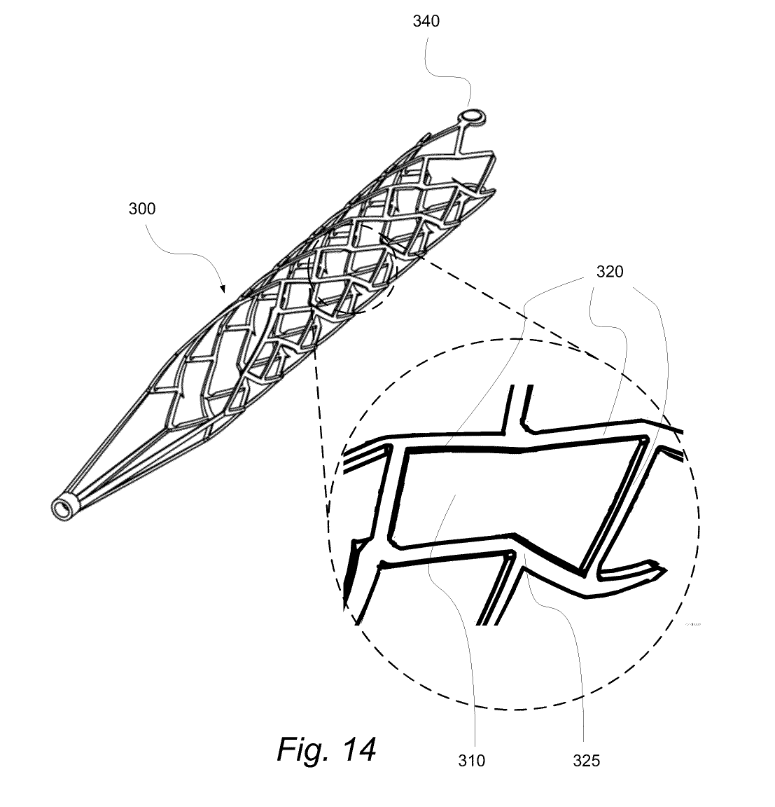

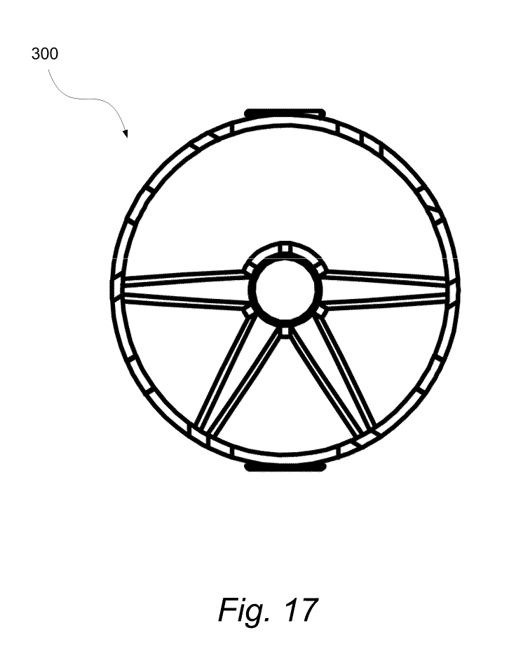

According to embodiments, other configurations may be provided for a stroke device. For example, as shown in FIGS. 14, 15, 16, and 17, stroke device 300 may have a radial geometry. As shown in FIG. 14, cells 310 may be defined by a plurality of struts 320 connected by bridges 325. As shown in FIG. 14, each strut 320 may connect at each of its ends at a bridge 325. Each bridge 325 may connect three struts. As further shown in FIG. 14, each open cell 310 may be defined by six struts 320, wherein the open cell 310 is substantially parallelogram-shaped.

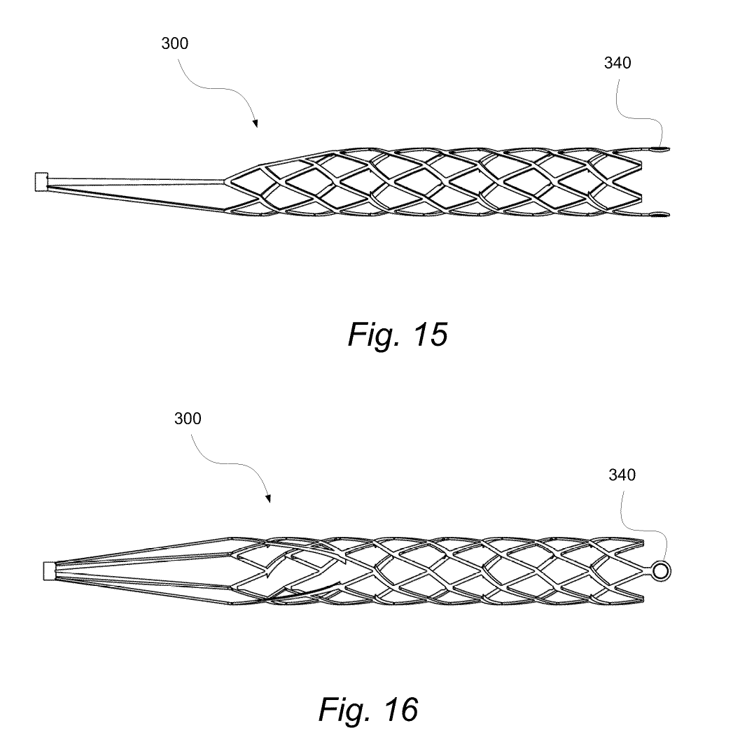

According to embodiments, as shown in FIG. 15, one end of stroke device 310 may include a tethering component for attachment to a catheter system. At the same end, stroke device 310 may provide an everted or scalloped geometry to facilitate recapture of stroke device 310 into the catheter.

While the method and agent have been described in terms of what are presently considered to be the most practical and preferred embodiments, it is to be understood that the disclosure need not be limited to the disclosed embodiments. It is intended to cover various modifications and similar arrangements included within the spirit and scope of the claims, the scope of which should be accorded the broadest interpretation so as to encompass all such modifications and similar structures. The present disclosure includes any and all embodiments of the following claims.

It should also be understood that a variety of changes may be made without departing from the essence of the invention. Such changes are also implicitly included in the description. They still fall within the scope of this invention. It should be understood that this disclosure is intended to yield a patent covering numerous aspects of the invention both independently and as an overall system and in both method and apparatus modes.

Further, each of the various elements of the invention and claims may also be achieved in a variety of manners. This disclosure should be understood to encompass each such variation, be it a variation of an embodiment of any apparatus embodiment, a method or process embodiment, or even merely a variation of any element of these.

Particularly, it should be understood that as the disclosure relates to elements of the invention, the words for each element may be expressed by equivalent apparatus terms or method terms—even if only the function or result is the same.

Such equivalent, broader, or even more generic terms should be considered to be encompassed in the description of each element or action. Such terms can be substituted where desired to make explicit the implicitly broad coverage to which this invention is entitled.

It should be understood that all actions may be expressed as a means for taking that action or as an element which causes that action.

Similarly, each physical element disclosed should be understood to encompass a disclosure of the action which that physical element facilitates.

Any patents, publications, or other references mentioned in this application for patent are hereby incorporated by reference. In addition, as to each term used it should be understood that unless its utilization in this application is inconsistent with such interpretation, common dictionary definitions should be understood as incorporated for each term and all definitions, alternative terms, and synonyms such as contained in at least one of a standard technical dictionary recognized by artisans and the Random House Webster's Unabridged Dictionary, latest edition are hereby incorporated by reference.

Finally, all referenced listed in the Information Disclosure Statement or other information statement filed with the application are hereby appended and hereby incorporated by reference; however, as to each of the above, to the extent that such information or statements incorporated by reference might be considered inconsistent with the patenting of this/these invention(s), such statements are expressly not to be considered as made by the applicant(s).

In this regard it should be understood that for practical reasons and so as to avoid adding potentially hundreds of claims, the applicant has presented claims with initial dependencies only.

Support should be understood to exist to the degree required under new matter laws—including but not limited to United States Patent Law 35 USC 132 or other such laws—to permit the addition of any of the various dependencies or other elements presented under one independent claim or concept as dependencies or elements under any other independent claim or concept.

To the extent that insubstantial substitutes are made, to the extent that the applicant did not in fact draft any claim so as to literally encompass any particular embodiment, and to the extent otherwise applicable, the applicant should not be understood to have in any way intended to or actually relinquished such coverage as the applicant simply may not have been able to anticipate all eventualities; one skilled in the art, should not be reasonably expected to have drafted a claim that would have literally encompassed such alternative embodiments.

Further, the use of the transitional phrase “comprising” is used to maintain the “open-end” claims herein, according to traditional claim interpretation. Thus, unless the context requires otherwise, it should be understood that the term “compromise” or variations such as “comprises” or “comprising”, are intended to imply the inclusion of a stated element or step or group of elements or steps but not the exclusion of any other element or step or group of elements or steps.

Such terms should be interpreted in their most expansive forms so as to afford the applicant the broadest coverage legally permissible.

1. An acute stroke system comprising, in combination:
a catheter with a guidewire effective for accessing of, and emplacement into, the cerebral vasculature;
an expandable stroke device tethered to the catheter and compressible within the catheter and radially expandable with a plurality of open cells defined by struts connected by bridges;
wherein the stroke device is configured to address a vascular clot by the flexion/extension of the stroke device upon expansion;
wherein each strut of the stroke device has a strut width and a strut thickness providing effective pinching stiffness and hoop stiffness for compressing the vascular clot to promote at least one of lysis, maceration, and removal thereof without compromising trackability of the stroke device.

2. The acute stroke system of claim 1, wherein the stroke device has an average COF per unit length across a diameter of 2.0 mm to 4.5 mm of between at least about 0.0025 N/mm and at least about 0.007 N/mm.

3. The acute stroke system of claim 1, wherein the stroke device has an average COF per unit length across a diameter of 2.0 mm to 4.5 mm of between at least about 0.0030 N/mm and at least about 0.0059 N/mm.

4. The acute stroke system of claim 1, wherein the stroke device has a COF range per unit length across a diameter of 2.0 mm to 4.5 mm of between at least about 0.00165 N/mm and at least about 0.0090 N/mm.

5. The acute stroke system of claim 1, wherein the stroke device has a RRF range per unit length across a diameter of 2.0 mm to 4.5 mm of between at least about 0.005 N/mm and at least about 0.016 N/mm.

6. The acute stroke system of claim 1, wherein the hoop stiffness of the pinching device is defined by the strut width, according to Eq. 7.

7. The acute stroke system of claim 1, wherein the pinching stiffness of the stroke device is defined by the strut thickness, according to Eq. 8.

8. The acute stroke system of claim 1, wherein the ratio of strut thickness to strut width is less than at least about 1.4.

9. The acute stroke system of claim 1, wherein the strut thickness is substantially equal to the strut width.

10. A stroke device comprising, in combination:
a structure having a substantially radial geometry, being tethered to a catheter, and having plurality of open cells defined by struts connected by bridges, said stroke device being radially expandable from a first state to a second state, wherein expansion to said second state provides effective maceration of a clot to which stroke device is applied during expansion to said second state; and
wherein each of said open cells has a length from about 0.120″ to about 0.250″ in said second state and a cell height from about 0.050″ to about 0.100″ in said second state, wherein said open cells are effective for promoting at least one of lysis, maceration, and removal of said clot.

11. The stroke device of claim 10, wherein each of said open cells has a substantially equal area of about 0.010 sq. inches to about 0.020 sq. inches.

12. The stroke device of claim 10, wherein the open cells vary throughout the stroke device having an area of between about 0.010 sq. inches and about 0.020 sq. inches.

13. An acute stroke system comprising, in combination:
a stroke device further comprising struts and bridges, wherein each strut has two ends, with each end connected to a bridge, wherein each bridge is connected to four struts, wherein the struts define open cells of the stroke device, each open cell being defined by four struts.

14. The stroke device of claim 13, wherein each strut is substantially linear.

15. The stroke device of claim 13, wherein each open cell is substantially diamond-shaped.

16. The stroke device of claim 13, wherein each bridge is substantially “C” shaped.

17. The stroke device of claim 13, wherein each bridge is substantially “S” shaped.

18. An acute stroke system comprising, in combination:
a stroke device further comprising struts and bridges, wherein each strut has two ends, with each end connected to a bridge, wherein each bridge is connected to three struts, wherein the struts define open cells of the stroke device, each open cell being defined by six struts.

19. The stroke device of claim 18, wherein each strut is substantially linear.

20. The stroke device of claim 18, wherein each open cell is substantially parallelogram-shaped.
